(19) United States
(12) Patent Application Publication
(10) Pub. No.: US 2026/0357698 A1
(43) Pub. Date: Nov. 12, 2026

(54) MINERAL COMPOSITION MAPPING BASED ON DENSITY MAPPING AND COMPOSITION ANALYSIS

(71) Applicant: Caterpillar Inc., Peoria, IL (US)

(72) Inventors: Lawrence A. Mianzo, Pittsburgh, PA (US); Tod A. Oblak, Pittsburgh, PA (US); Carl Hendricks, Mosman Park (AU)

(73) Assignee: Caterpillar Inc., Peoria, IL (US)

(21) Appl. No.: 19/203,821

(22) Filed: May 9, 2025

(51) Int. Cl. E21B 49/02; G01N 33/24; G01V 5/08

(52) U.S. Cl. CPC E21B 49/02; G01N 33/24; G01V 5/08

(57) ABSTRACT

A method and a system disclosed herein are directed to generating a three dimensional (3D) mineral composition map based on a 3D density map. The method includes generating a 3D density map based on cosmic ray muon (CRM) tomography; determining bounding surfaces around a region of interest (ROI) in the 3D density map based on at least one of density ranges selected for the ROI or historical density ranges associated with expected mineral compositions; determining a plurality of drilling locations based on the ROI; determining mineral compositions of core samples obtained from core sample locations, the core sample locations associated with the plurality of drilling locations in real-time utilizing hyperspectral analysis; and generating a 3D mineral composition map based on the 3D density map and the mineral compositions of the core samples.

### TECHNICAL FIELD

The present disclosure relates to subsurface mineral composition mapping, and more specifically to mapping of mineral composition subsurface by combining density mapping of an area with composition analysis at selected locations within the area.

### BACKGROUND

To locate and determine mineral compositions at various depths of a surface mining site for a blasthole drilling based surface mining, a number of core sample holes over the mining site is drilled and core samples are extracted from various areas of the surface mining site and the core samples are sent out a laboratory for analysis. This process of drilling, extracting, and analyzing to locate and determine mineral compositions of the surface mining site is time consuming and affect the overall mining operations. For example, the core drilling may be performed days, weeks, or even months ahead of the blasthole drilling to decide on mining a region of interest (ROI) if core assaying indicates that the material in the ROI is worthwhile extracting.

One system for forming a three-dimensional density distribution image is described in U.S. Patent No. 7,531,791 (“the ’791 patent”). The ’791 patent describes a system and method for detecting cosmic ray muon (CRM) flux in a region and processing the data to form a three-dimensional (3D) density distribution image corresponding to the region for identifying concentrations of higher (or lower) density mineral deposits. However, different mineral compositions can have the same density, a map of describing a uniform density region does not guarantee that the uniform density region contains the same mineral compositions. While useful in creating a 3D density map utilizing CRM tomography, the ’791 patent does not describe determining mineral compositions.

Examples of the present disclosure are directed to overcoming deficiencies of such systems.

### SUMMARY

In an aspect of the present disclosure, a method for generating a three dimensional (3D) mineral composition map is provided. The method includes generating a 3D density map based on cosmic ray muon (CRM) tomography; determining bounding surfaces around a region of interest (ROI) in the 3D density map based on at least one of: density ranges selected for the ROI or historical density ranges associated with expected mineral compositions; determining a plurality of drilling locations based on the ROI; determining mineral compositions of core samples obtained from core sample locations, the core sample locations associated with the plurality of drilling locations in real-time utilizing hyperspectral analysis; and generating a 3D mineral composition map based on the 3D density map and the mineral compositions of the core samples.

In another aspect of the present disclosure, a system includes one or more processors and a memory coupled to the one or more processors, the memory storing computer-readable instructions that, when executed by the one or more processors, cause the one or more processors to perform operations. The operations include generating a 3D density map based on cosmic ray muon (CRM) tomography; determining bounding surfaces around a region of interest (ROI) in the 3D density map based on at least one of: the density ranges selected for the ROI or historical density ranges associated with expected mineral compositions; determining a plurality of drilling locations based on the ROI; determining mineral compositions of core samples obtained from core sample locations, the core sample locations associated with the plurality of drilling locations in real-time utilizing hyperspectral analysis; and generating a 3D mineral composition map based on the 3D density map and the mineral compositions of the core samples.

In yet another aspect of the present disclosure, non-transitory computer-readable storage media is provided. The non-transitory computer-readable storage media stores computer-readable instructions that, when executed by one or more processors, cause the one or more processors to perform operations. The operations include generating a 3D density map based on cosmic ray muon (CRM) tomography; determining bounding surfaces around a region of interest (ROI) in the 3D density map based on at least one of: the density ranges selected for the ROI or historical density ranges associated with expected mineral compositions; determining a plurality of drilling locations based on the ROI; determining mineral compositions of core samples obtained from core sample locations, the core sample locations associated with the plurality of drilling locations in real-time utilizing hyperspectral analysis; and generating a 3D mineral composition map based on the 3D density map and the mineral compositions of the core samples.

### BRIEF DESCRIPTION OF DRAWINGS

The detailed description references the accompanying figures. In the figures, the left-most digit of a reference number identifies the figure in which the reference number first appears. The same reference numbers indicate similar or identical items.

FIG. 1 is an example density map of a surface mining site.

FIG. 2 illustrates example drilling locations based on the density map for obtaining core samples from regions of interest.

FIG. 3 is a mineral composition map generated based on the density map using mineral composition analysis techniques.

FIG. 4 is a flow chart illustrating an example process for generating a 3D mineral composition map from a density map using the mineral composition analysis techniques.

FIG. 5 is a flow chart illustrating an example sub-process of one of the blocks of FIG. 4.

FIG. 6 is a flow chart illustrating another example sub-process of one of the blocks of FIG. 4.

FIG. 7 is a block diagram of an example system for generating a 3D mineral composition map.

### DETAILED DESCRIPTION

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value.

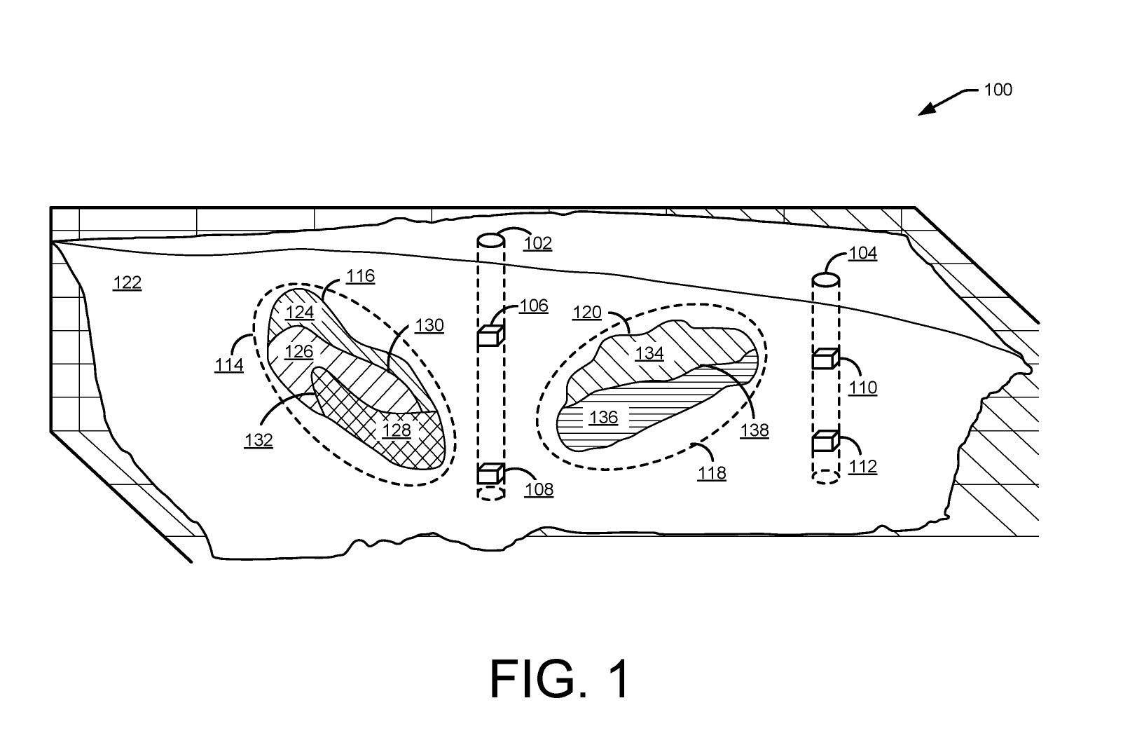

FIG. 1 is a cutaway view of an example 3D density map 100 of a surface mining site generated using cosmic ray muon (CRM) tomography, or simply muon tomography. Muon tomography uses a grid of muon detectors/transceivers (DeTXs) inserted into the ground to detect overlapping zones of density image maps. In this example, two core sample holes, a first core sample hole 102 and a second core sample hole 104, are shown with four DeTXs 106, 108, 110, and 112, inserted at various depths. The core sample holes 102 and 104 may be, or may have been, drilled to extract core samples, which are typically sent out a laboratory for analysis. Using tomography, the zone “slice” image maps can be used to reconstruct a 3D geographic map of the densities of the subsurface terrain as the 3D density map 100 shows. In this example, the 3D density map 100 includes a first region of interest (ROI) 114 bounded by a first bounding surface 116 and a second ROI 118 bounded by a second bounding surface 120 having densities within a range of interest associated with known mineral compositions. Other areas 122 are regions of varying densities outside of the range of interest.

Based on the density variation within the first ROI 114, the first ROI 114 may be further divided into three sub-regions including a first sub-region 124, a second sub-region 126, and a third sub-region 128. The first sub-region 124 may be separated from the second sub-region 126 by a first sub-bounding surface 130, and the second sub-region 126 may be separated from the third sub-region 128 by a second sub-bounding surface 132. Based on the density variation within the second ROI 118, the second ROI 118 may be further divided into two sub-regions including a fourth sub-region 134 and a fifth sub-region 136. The fourth sub-region 134 may be separated from the fifth sub-region 136 by a third sub-bounding surface 138. The bounding surfaces, such as the first bounding surface, 116, the second bounding surface 120, the first sub-bounding surface 130, the second sub-bounding surface 132, and the third sub-bounding surface 138, may be determined based on density ranges selected for the first ROI 114 and the second ROI 118, and/or historical data associated with density ranges of expected mineral compositions.

While the densities of the first ROI 114 and the second ROI 118 may fall within the range of interest, the 3D density map 100 does not provide actual mineral compositions of the first ROI 114 or the second ROI 118. Different mineral compositions may produce densities within the range of interest. Therefore, further exploration may be required. Because the 3D density map 100 provides regions of interest, such as the first ROI 114 and the second ROI 118, a plurality of drilling locations for core sample holes for sampling materials from within the first ROI 114 and the second ROI 118 may be determined, and samples from appropriate depths, such as depths corresponding to the first sub-region 124, the second sub-region 126, the third sub-region 128, fourth sub-region 134, and the fifth sub-region 136, from the core sample holes at the plurality of locations, may be collected for analysis. Therefore, the 3D density map 100 may allow avoiding unnecessary core sample holes in the other areas 122, where no mineral compositions having densities within the range of interest exist, and save time and resources. For surface mining and mineral recovery, a subset of core sample holes from a pattern of core sample holes may be selected based on a density map, such as the 3D density map 100, corresponding to the areas over the first ROI 114 and the second ROI 118, and samples from appropriate depths, such as depths corresponding to the first sub-region 124, the second sub-region 126, the third sub-region 128, fourth sub-region 134, and the fifth sub-region 136, may be collected for analysis.

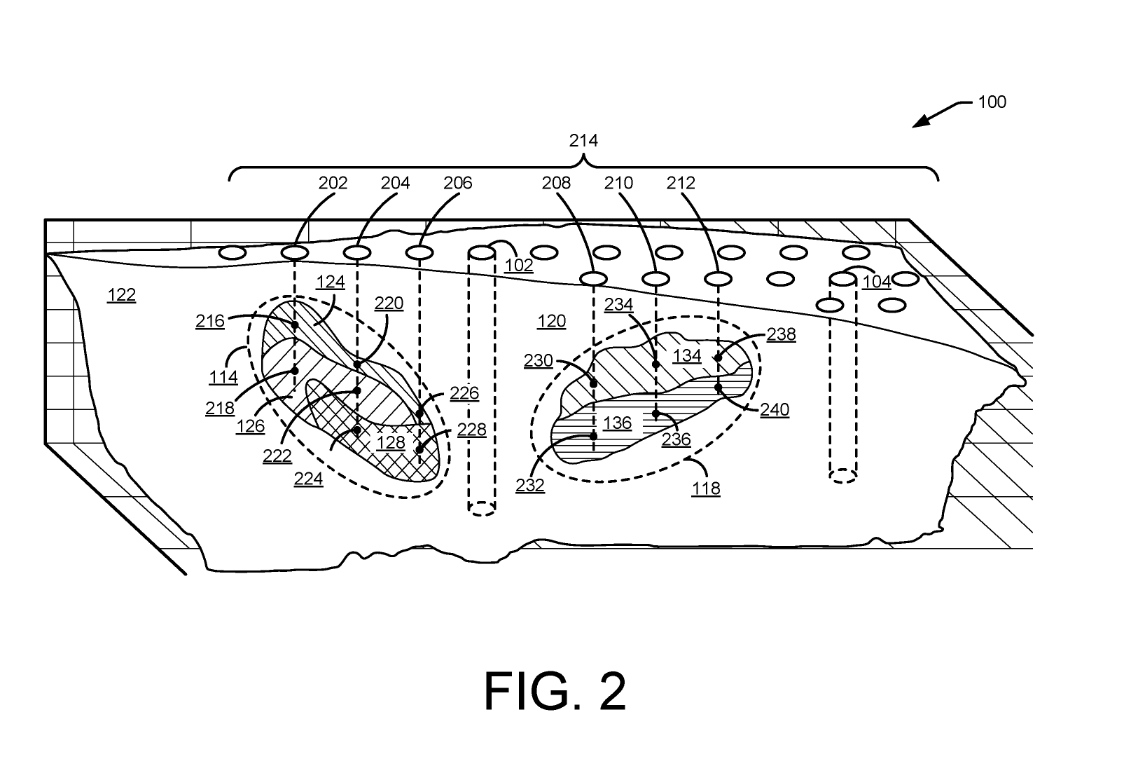

FIG. 2 illustrates example drilling locations based on the 3D density map 100 for obtaining core samples from regions of interest, such as the first ROI 114 and the second ROI 118. Mineral composition analysis techniques may be performed on core samples and/or drilled particles obtained from the locations of the first ROI 114 and the second ROI 118 from the 3D density map 100, and a plurality of drilling locations for core sample holes, such as, but not limited to, six core sample holes 202, 204, 206, 208, 210, and 212 shown in this example, may be determined or selected for sampling materials from within the first ROI 114 and the second ROI 118. While core sample holes are normally spaced between 50m and 500m apart, these core sample hole 202, 204, 206, 208, 210, and 212 may be selectively located over the first ROI 114 and the second ROI 118. Additionally, or alternatively, a subset of blastholes selected from a pattern, such as a predetermined grid, of blasthole drilling 214 for surface mining and mineral recovery as shown in this example, may be utilized for performing hyperspectral analysis as described below. At each location of the plurality of drilling locations, a core sample hole may be drilled to a depth, or depths, reaching one or more sub-regions.

For example, at the core sample hole 202, a first core sample may be obtained at a first core sample location 216 within the first sub-region 124 of a first depth, and a second core sample may be obtained at a second core sample location 218 within the second sub-region 126 of a second depth. At the core sample hole 204, a third core sample may be obtained at a third core sample location 220 within the first sub-region 124 of a third depth, a fourth core sample may be obtained at a fourth core sample location 222 within the second sub-region 126 of a fourth depth, and a fifth core sample may be obtained at a fifth core sample location 224 within the third sub-region 128 of a fifth depth. At the core sample hole 206, a sixth core sample may be obtained at a sixth core sample location 226 within the first sub-region 124 of a sixth depth, and a seventh core sample may be obtained at a seventh core sample location 228 within the third sub-region 128 of a seventh depth. At the core sample hole 208, an eighth core sample may be obtained at an eighth core sample location 230 within the fourth sub-region 134 of an eighth depth, and a ninth core sample may be obtained at a ninth core sample location 232 within the fifth sub-region 136 of a ninth depth. At the core sample hole 210, a tenth core sample may be obtained at a tenth core sample location 234 within the fourth sub-region 134 of a tenth depth, and an eleventh core sample may be obtained at an eleventh core sample location 236 within the fifth sub-region 136 of an eleventh depth. At the core sample hole 212, a twelfth core sample may be obtained at a twelfth core sample location 238 within the fourth sub-region 134 of a twelfth depth, and a thirteenth core sample may be obtained at a thirteenth core sample location 240 within the fifth sub-region 136 of a thirteenth depth.

While the core samples may typically be sent to a laboratory for analysis and determination of mineral contents, mineral contents of core samples may also be analyzed in real-time, for example, by performing hyperspectral analysis on drilled particles, corresponding to core sample locations of the core samples, exiting a drilled hole during the drilling process to obtain the core sample. By determining the mineral contents while drilling for, and extracting, the core samples in real-time, it is possible to determine whether a sub-region within an ROI, such as the first sub-region 124 within the first ROI 114, contains expected mineral compositions and/or other mineral compositions having the same or similar density as the expected mineral compositions. For example, if mineral contents from the core sample locations 216, 220, and 226 within the first sub-region 124 match a first expected mineral compositions, then the first expected mineral compositions may be assigned to the first sub-region 124. While in this example, three core sample locations are used for illustration, a larger number of core samples, or core sample locations, may be used in an actual application to determine mineral compositions of a sub-region, and a confidence level, such as a composition confidence level, may be utilized to determine whether a sub-region can be considered to contain expected mineral compositions.

For example, 20 core samples, or mineral compositions results from hyperspectral analysis while obtaining the core samples, from 20 core sample locations within the same sub-region may be used to determine whether the sub-region contains the expected mineral compositions. A threshold composition confidence level may be set, for example, to 80%, i.e., if 16 or more core samples, or hyperspectral mineral compositions results, out of 20 match the expected mineral compositions within a preselected range, then the sub-region from which the 20 core samples are taken may be considered to contain the expected mineral compositions and may be assigned the expected mineral compositions to the ROI. However, if fewer than 16 core samples, or hyperspectral mineral compositions results, out of 20 match the expected mineral compositions within the preselected range, then the sub-region from which the 20 core samples, or hyperspectral mineral compositions results, are taken may be considered to contain other mineral compositions in addition to the expected mineral compositions, and a sub-bounding surface, or a bounding surface, associated with the sub-region may need to be redrawn. To redraw the sub-bounding surface, one or more drilling locations different from the plurality of drilling locations may be determined based on the ROI. At each drilling location of the one or more drilling locations, a new core sample may be obtained by drilling to a preselected depth within the ROI and analyzed in real-time, and the mineral compositions of the new core sample may be determined. Based on the mineral compositions of the new core samples from the one or more drilling locations, a revised sub-bounding surface, or a revised bounding surface may be drawn.

If a preselected number of core samples, or hyperspectral mineral compositions results, from a cluster, i.e., neighboring core sample locations, fail to match the expected mineral compositions within the preselected range, then the area of the cluster may be considered not to contain the expected mineral compositions, and the sub-bounding surface associated with the sub-region may need to be redrawn. Additionally, or alternatively, a cutoff composition confidence level may be set, for example, to 25%, i.e., if 15 or more core samples, or hyperspectral mineral compositions results, out of 20 fail to match the expected mineral compositions within the preselected range, then the sub-region from which the 20 core samples, or hyperspectral mineral compositions results, are taken may be considered not to contain the expected mineral compositions. A revised sub-bounding surface, or a revised bounding surface, may then be drawn according to the process of redrawing the sub-bounding/bounding surface as described above.

Additionally, or alternatively, a drilling force applied for drilling a blasthole may be monitored to determine whether the drilling force applied is within an acceptable drilling force range for expected mineral compositions at the preselected depth. The drilling force monitored may include applied torque, pulldown pressure, vibration, and revolution-per-minute (rpm) of the drill being utilized. For example, 20 drilling forces applied at 20 different core sample locations within the same sub-region may be used to determine whether the sub-region contains the expected mineral compositions. A threshold drilling force confidence level may be set, for example, to 80%, i.e., if 16 drilling forces applied out of 20 drilling forces applied are within an acceptable drilling force range for the expected mineral compositions at a preselected depth, then the sub-region may be considered to contain the expected mineral compositions and may be assigned the expected mineral compositions to the ROI. However, if fewer than16 drilling forces applied out of 20 drilling forces applied are within the acceptable drilling force range, then the sub-region may be considered to contain other mineral compositions in addition to the expected mineral compositions, and a sub-bounding surface associated with the sub-region may need to be redrawn. To redraw the sub-bounding surface, one or more drilling locations different from the plurality of drilling locations may be determined based on the ROI. At each drilling location of the one or more drilling locations, a drilling force applied may be monitored for drilling at a preselected depth, and whether the drilling force applied is within the acceptable drilling force range for the expected mineral compositions may be determined. Based on the drilling forces applied at the one or more drilling locations, a revised sub-bounding surface, or a revised bounding surface may be drawn. Additionally, hyperspectral analysis may be performed on drilled particles exiting a drilled blasthole as the blasthole is being drilled to confirm the material compositions of the ROI or the sub-region.

If a preselected number of drilling forces applied from a cluster, i.e., neighboring core sample locations, fail to be within the acceptable drilling force range, then the area of the cluster may be considered not to contain the expected mineral compositions, and the sub-bounding surface associated with the sub-region may need to be redrawn. Additionally, or alternatively, a cutoff drilling force confidence level may be set, for example, to 25%, i.e., if 15 or more drilling forces applied out of 20 drilling forces applied fail to be within the acceptable drilling force range associated with the expected mineral composition, then the sub-region may be considered not to contain the expected mineral compositions. A revised sub-bounding surface, or a revised bounding surface, may then be drawn according to the process of redrawing the sub-bounding/bounding surface as described above.

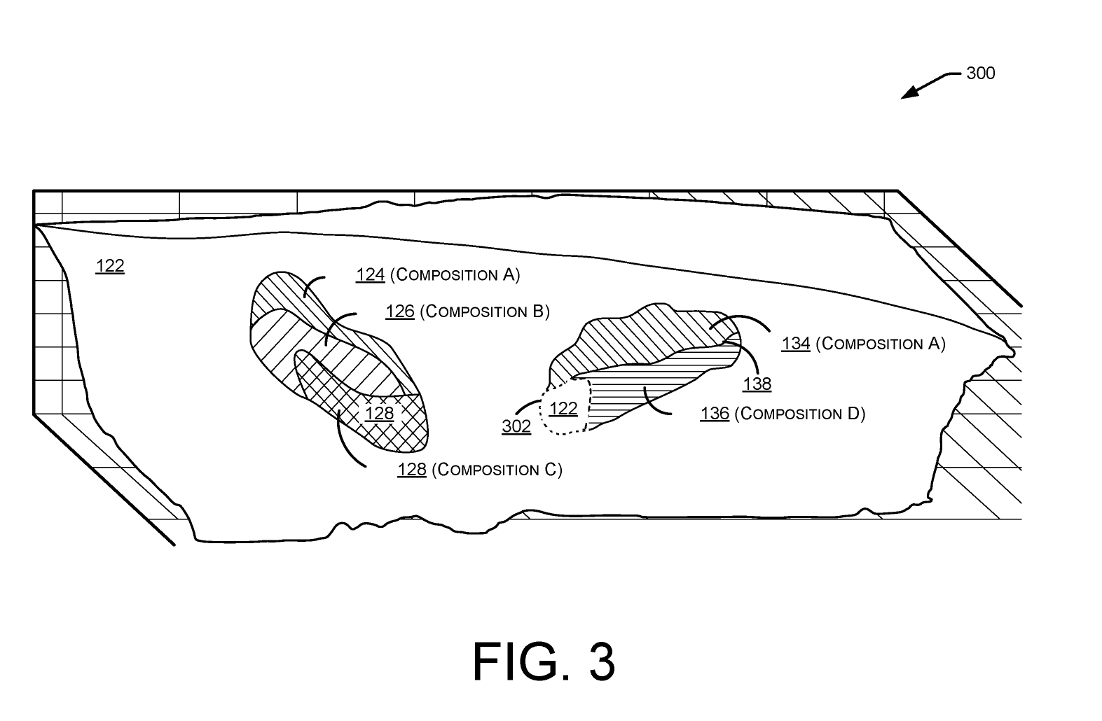

FIG. 3 is an example 3D mineral composition map 300, based on rock types, generated from the 3D density map 100 using the mineral composition analysis techniques described above with reference to FIG. 2. In this example, the third sub-bounding surface 138 is revised as a result of the core samples and/or the drill forces applied in the fifth sub-region 136. A resulting sixth sub-region 302 shown with a dotted line is now assigned as a part of the other areas 122 not containing a material composition of interest. The first sub-region 124 and the fourth sub-region 134 may be assigned the same mineral compositions, such as granodiorite with chalcopyrite mineralization (shown as composition A), the second sub-region 126 may be assigned as a quartz porphyry with strong clay alteration (shown as composition B), the third sub-region 128 may be assigned as a quartz porphyry with weak clay alteration (shown as composition C), and the fifth sub-region 136 may be assigned as a quartz porphyry with moderate clay alteration (shown as composition D).

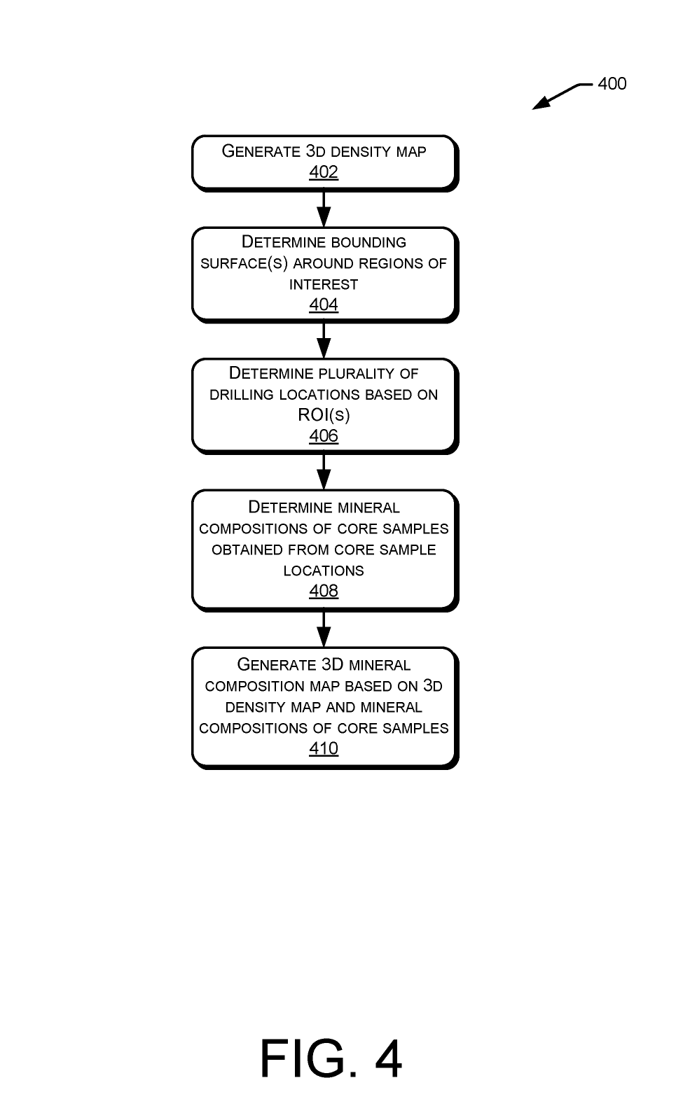

FIG. 4 is a flow chart illustrating an example process 400 for generating a 3D mineral composition map, such as the mineral composition map 300, from a density map, such as the 3D density map 100 generated using tomography, using the mineral composition analysis techniques described above with reference to FIGS. 1-3. The example process 400 is illustrated as a collection of steps in a logical flow diagram, which represents operations that can be performed, for example, by one or more processors of a system, to generate a 3D mineral composition map as described above with reference to FIGS. 1-3. The order in which the operations are described is not intended to be construed as a limitation, and any number of the described steps can be combined and performed in any order, in parallel, or simultaneously to implement the process.

At block 402, a 3D density map, such as the 3D density map 100, may be generated based on tomography. The 3D density map 100 may be generated based on data obtained, for example, by utilizing cosmic ray muon (CRM) tomography with a grid of muon detectors/transceivers (DeTXs) 106, 108, 110, and 112, as described above with reference to FIG. 1. At block 404, bounding surfaces, such as the first bounding surface 116 and the second bounding surface 120, around one or more regions of interest (ROIs), such as the first ROI 114 and the second ROI 118, in the 3D density map 100 may be determined. A bounding surface may be determined based on density ranges selected for an ROI, such as a density range between 1800 kg/m³ and 2300 kg/m³ for a quartz porphyry with weak clay alteration. Additionally, or alternative, the density ranges may be historical density ranges associated with expected mineral compositions, such as a density range between 2600 kg/m³ and 2900 kg/m³ for an expected mineral composition at the mining site, for example, a granodiorite. The first ROI 114 and the second ROI 118 may be further divided into sub-regions, such as the first sub-region 124, the second sub-region 126, the third sub-region 128, the fourth sub-region 134, and the fifth sub-region 136, by selecting narrower ranges of densities and determining sub-bounding surfaces, such as the first sub-bounding surface 130 and the second sub-bounding surface 132 within the first ROI 114, and the third sub-bounding surface 138 within the second ROI 118, as discussed above with reference to FIG. 2.

At block 406, a plurality of drilling locations based on the ROI, such as the first ROI 114 and the second ROI 118, may be determined. Because the locations of the first ROI 114 and the second ROI 118 are known from the 3D density map 100, the plurality of drilling locations, such as for the core sample holes, 202, 204, 206, 208, 210, and 212 as shown in FIG. 2, may be located over the first ROI 114 and the second ROI 118 to reach all sub-regions, as the first sub-region 124, the second sub-region 126, the third sub-region 128, the fourth sub-region 134, and the fifth sub-region 136, to obtain core samples from each sub-region. Instead of traditionally drilling a large number of core sample holes and obtaining core samples at various depths, drilling core sample holes only over the ROIs and collecting core samples at known depths based on the 3D density map save time and resources over the traditional process. At block 408, mineral compositions of core samples obtained from core sample locations may be determined. For example, the core samples may be drilled particles exiting a drilled hole, i.e., a core sample hole, corresponding to a core sample location, and mineral contents of the drilled particles may be analyzed in real-time by performing hyperspectral analysis on the drilled particles during the drilling process to obtain the core sample. The core sample locations may be associated with the plurality of drilling locations. The core sample locations may be determined, or selected, to be within sub-regions, i.e., at preselected depths, that a bore hole at each location of the plurality of drilling locations is expected to encounter. Additionally, or alternatively, the plurality of drilling locations selected from a pattern of blasthole drilling, such as the predetermined grid for blasthole drilling 214, for surface mining and mineral recovery, may be utilized to perform hyperspectral analysis on drilled particles exiting a drilled hole during the blasthole drilling as discussed above with reference to FIG. 2. For example, the core sample locations for the core sample hole 204 may include the third core sample location 220 for a core sample from the first sub-region 124, the fourth core sample location 222 for a core sample from the second sub-region 126, and the fifth core sample location 224 for a core sample from the third sub-region 128, as shown in FIG. 2. While each core sample may be sent to a laboratory, which may be off-site, for mineral composition analysis, each core sample may be analyzed and determined in real-time using hyperspectral analysis (hyperspectroscopy) as each core sample is obtained. Based on the mineral compositions of the core samples and the 3D density map 100, a 3D mineral composition map, such as the 3D mineral composition map 300 may be generated at block 410. For example, the mineral compositions of core samples from the core sample locations 216, 220, and 226 within the first sub-region 124 match expected mineral compositions of the core sample location within an acceptable composition range, and therefore, the sub-region 124 may be determined to contain the expected mineral compositions. Similarly, mineral compositions of each sub-region may be determined based on the mineral compositions of the core samples from core sample locations within the corresponding sub-regions.

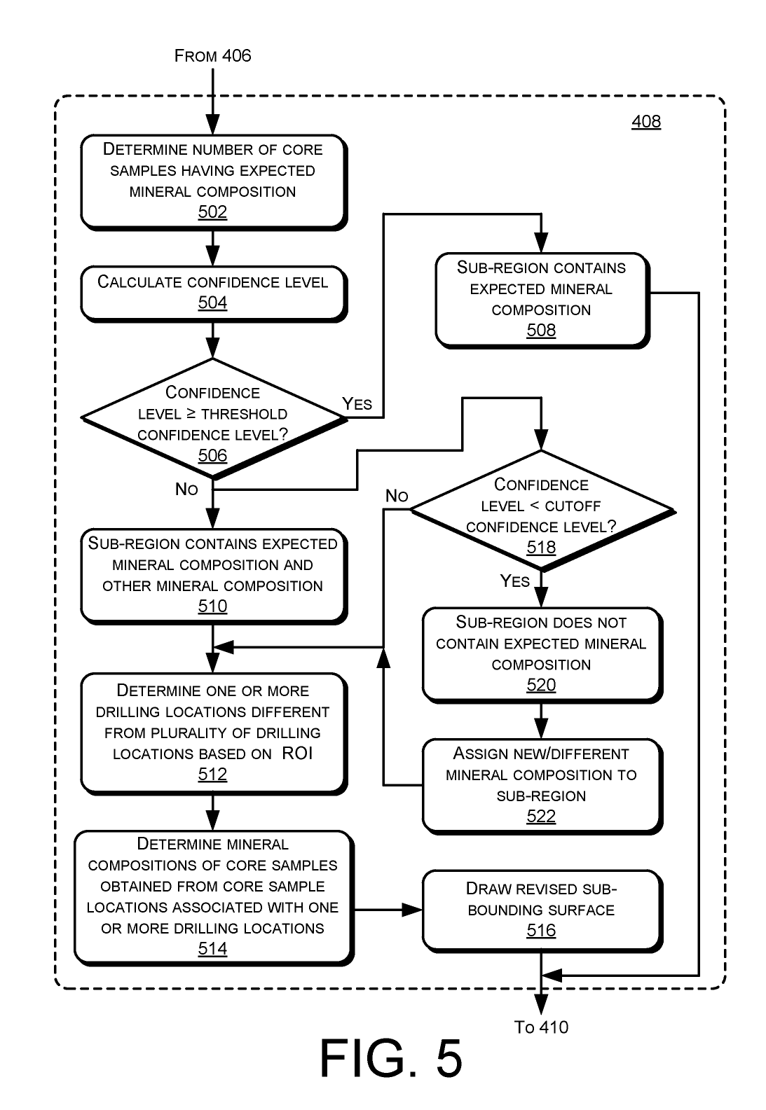

FIG. 5 is a flow chart illustrating an example sub-process of block 408 of FIG. 4. Core samples may be physical core samples extracted from a core sample hole drilled or mineral contents analyzed in real-time by performing hyperspectral analysis on drilled particles exiting the drilled core sample hole during the drilling process to obtain the core sample. While three core samples are used in block 408 above as an example, a larger number of core samples may be used in an actual application to determine mineral compositions of a sub-region, and a confidence level, such as a composition confidence level, may be utilized to determine whether a sub-region can be considered to contain expected mineral compositions. For example, 20 core samples from 20 core sample locations within the same sub-region may be used to determine whether the sub-region contains the expected mineral compositions.

At block 502, a number of core samples, from a predetermined number of core sample locations within the same sub-region, matching expected mineral compositions within a preselected range may be determined. At block 504, the composition confidence level based on a core sample size and a number of core samples with the mineral compositions matching corresponding expected mineral compositions may be calculated. For example, for a core sample size of 20 with 16 core samples matching the expected mineral compositions, the composition confidence level is 80% (16/20 = 0.8). At block 506, whether the composition confidence level is greater than or equal to a threshold confidence level, such as a threshold composition confidence level, may be determined. If the composition confidence level is greater than or equal to the threshold composition confidence level (“YES” branch), then the sub-region, from which the core samples are obtained, may be considered, or determined, to contain the expected mineral compositions at block 508. For example, for the core sample size of 20 and the threshold composition confidence level set to 80%, the sub-region, from which the 20 core samples are taken, may be considered, or determined, to contain the expected mineral compositions if 16 or more core samples match the expected mineral compositions within the preselected range. However, if fewer than 16 core samples out of 20 core samples match the expected mineral compositions within the preselected range (“No” branch), then the sub-region may be considered to contain other mineral compositions in addition to the expected mineral compositions at block 510, and a sub-bounding surface, or a bounding surface, associated with the sub-region may need to be redrawn. To redraw the sub-bounding surface, one or more drilling locations different from the plurality of drilling locations may be determined based on the ROI at block 512. Mineral compositions of core samples obtained from core sample locations associated with the one or more drilling locations may be determined at block 514. For example, at each drilling location of the one or more drilling locations, a new core sample may be obtained by drilling to a corresponding depth associated with each core sample location within the ROI and analyzed in real-time, and the mineral compositions of the new core sample may be determined. Based on the mineral compositions of the new core samples from the one or more drilling locations, a revised sub-bounding, or bounding, surface, or a revised bounding surface may be drawn at block 516.

If more than a preselected percentage of core samples from a cluster, i.e., neighboring core sample locations, fails to match the expected mineral compositions within the preselected range, then the area of the cluster may be considered not to contain the expected mineral compositions, and the sub-bounding surface associated with the sub-region may need to be redrawn. Additionally, or alternatively, a cutoff composition confidence level may be set. At block 518 after the composition confidence level is determined to be less than the threshold composition confidence level at block 506 (“No” branch), whether the composition confidence level is less than a cutoff confidence level, such as a cutoff composition confidence level, may be determined. For example, for the cutoff composition confidence level of 25%, i.e., if 5 or fewer core samples out of 20 core samples match the expected mineral compositions within the preselected range (“Yes” branch), then the sub-region, from which the 20 core samples are taken, may be considered not to contain the expected mineral compositions at block 520. A new and/or different mineral compositions may then be assigned to the sub-region at block 522 as shown FIG. 3 as sixth sub-region 302 belonging to the other areas 122. If the composition confidence level is determined to be greater than the cut off composition confidence level (“No” branch) at block 518, then the process may loop back to block 512.

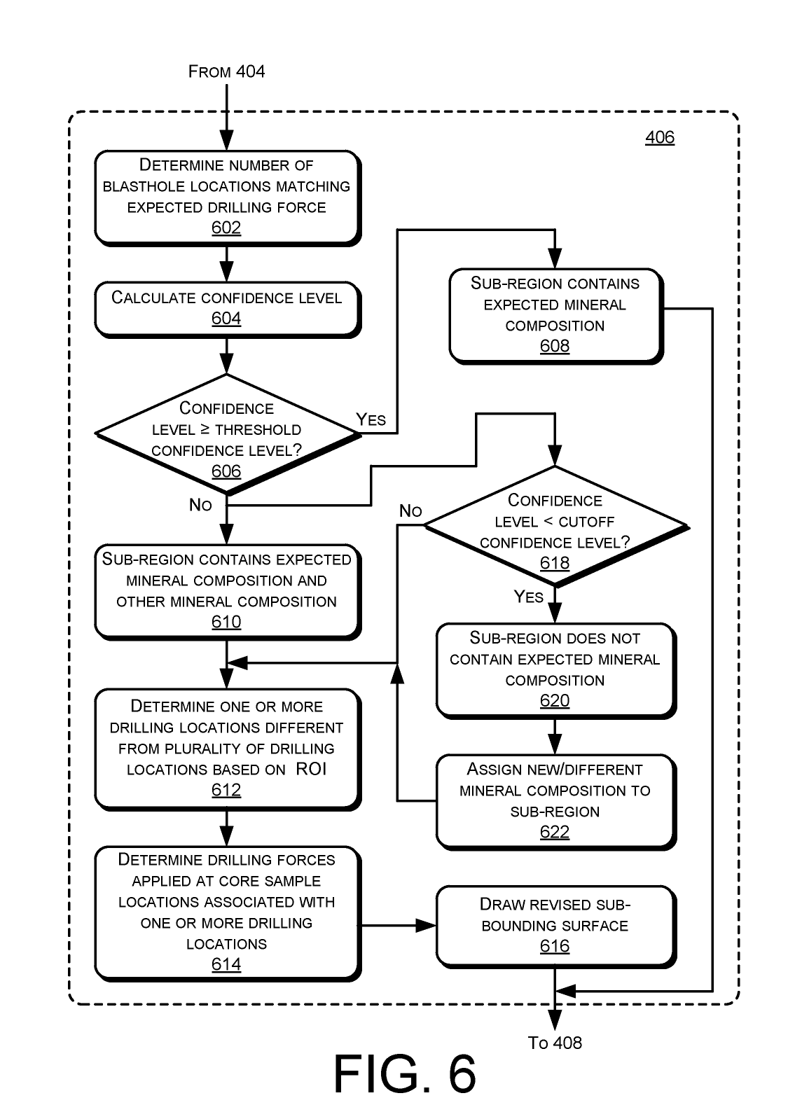

FIG. 6 is a flow chart illustrating additional or alternative example sub-process of block 406 of FIG. 4. For example, drilling forces applied may be monitored at 20 core sample locations within the same sub-region, and a confidence level, such as a drilling force confidence level, may be used to determine that the drilling force applied are consistent with the expected mineral compositions contained in the sub-region.

At block 602, a number of blasthole locations, within the same sub-region where drilling forces applied matching a drilling force expected for the expected mineral compositions within an acceptable drilling force range, may be determined. At block 604, a confidence level, such as a drilling force confidence level, based on the number of core sample locations and a number of core sample locations with the drilling force applied matching the drilling force expected, may be calculated. For example, for a core sample location size of 20 with 16 core sample locations matching the expected drilling force, the drilling force confidence level is 80% (16/20 = 0.8). At block 606, whether the drilling force confidence level is greater than or equal to a threshold confidence level, such as a threshold drilling force confidence level, may be determined. If the drilling force confidence level is greater than or equal to the threshold drilling force confidence level (“YES” branch), then the sub-region including the 20 core sample locations may be considered, or determined, to contain the expected mineral compositions at block 608, and the process may proceed to block 410. For example, for the core sample location size of 20 and the threshold drilling force confidence level set to 80%, the sub-region including the 20 core sample locations may be considered, or determined, to contain the expected mineral compositions if 16 or more core sample locations match the expected drilling force within the acceptable drilling force range. However, if fewer than 16 core sample locations out of 20 core sample locations match the expected drilling force within the acceptable drilling force range (“No” branch), then the sub-region may be considered to contain other mineral compositions in addition to the expected mineral compositions at block 610, and a sub-bounding surface, or a bounding surface, associated with the sub-region may need to be redrawn. To redraw the sub-bounding surface, one or more drilling locations different from the plurality of drilling locations may be determined based on the ROI at block 612. Drilling forces applied at core sample locations associated with the one or more drilling locations may be determined at block 614. For example, at each drilling location of the one or more drilling locations, a drilling force applied at each core sample location within the ROI may be monitored and determined. Based on the drilling force applied at the core sample location, the material composition of the sub-region at the core sample location may be determined and, a revised sub-bounding, or bounding, surface, or a revised bounding surface may be drawn at block 616, and the process may proceed to block 408.

If more than a preselected percentage of the drilling forces applied from a cluster, i.e., neighboring core sample locations, fails to match the drilling force expected for the expected mineral compositions within the acceptable drilling force range, then the area of the cluster may be considered not to contain the expected mineral compositions, and the sub-bounding surface associated with the sub-region may need to be redrawn. Additionally, or alternatively, a cutoff drilling force confidence level may be set. At block 618 after the drilling force confidence level is determined to be less than the threshold drilling force confidence level at block 606 (“No” branch), whether the drilling force confidence level is less than the cutoff composition confidence level, may be determined. For example, for the cutoff composition confidence level of 25%, i.e., if 5 or fewer core sample locations out of 20 core sample locations match the drilling force expected for the expected mineral compositions within the acceptable drilling force range (“Yes” branch), then the sub-region including the 20 core sample locations may be considered not to contain the expected mineral compositions at block 620. A new and/or different mineral compositions may then be assigned to the sub-region at block 622 as shown in FIG. 3 as sixth sub-region 302 belonging to the other areas 122. If the drilling force confidence level is determined to be greater than the cut off composition confidence level (“No” branch) at block 618, then the process may loop back to block 612.

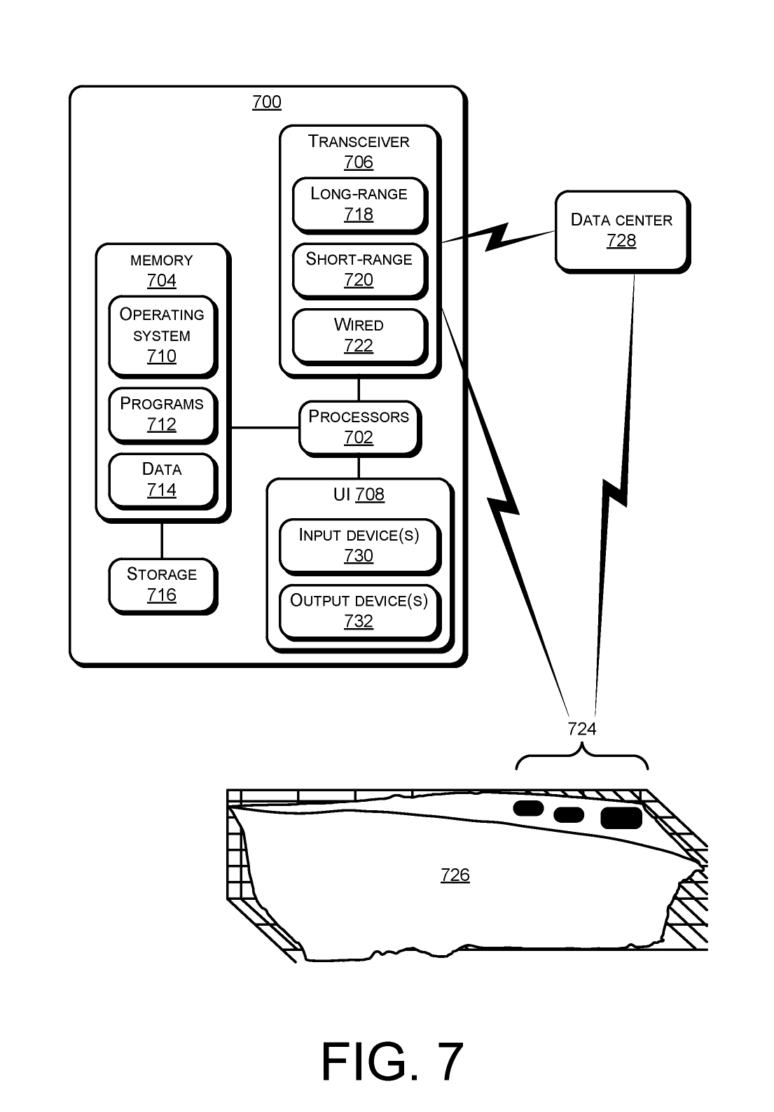

FIG. 7 is a block diagram of an example system 700 for generating a 3D mineral composition map based on techniques described above with reference to FIGS. 1-6. In various embodiments, the system 700 may include one or more processors (processors) 702, memory 704 coupled to the processors 702, a transceiver 706 coupled to the processors 702, and a user interface (UI) 708 coupled to the processors 702.

The processors 702 can be a central processing unit (CPU), a graphics processing unit (GPU), or both CPU and GPU, or any other type of processing unit. Each of the one or more processors 702 may have numerous arithmetic logic units (ALUs) that perform arithmetic and logical operations, as well as one or more control units (CUs) that extract instructions and stored content from processor cache memory, and then executes these instructions by calling on the ALUs, as necessary, during program execution. The processors 702 may also be responsible for executing all computer applications stored in the memory 704.

Depending on the exact configuration and type of the system 700, the memory 704 may be volatile (such as RAM), non-volatile (such as ROM, flash memory, etc.) or some combination of both. The memory 704 may include, or store, an operating system 710, one or more program modules 712, and may include program data 714. The system 700 may also include additional data storage devices (removable and/or non-removable) such as, for example, a flash drive, an SD card, magnetic disks, optical disks, or tape. Such additional storage is illustrated in FIG. 7 as a storage 716.

Non-transitory computer-readable storage media of the system 700, such as the memory 704 and the storage 716, may include volatile and nonvolatile, removable and non-removable media implemented in any method or technology for storage of information, such as computer-readable instructions, data structures, program modules, or other data. The memory 704 and storage 716 are all examples of computer-readable storage media. Non-transitory computer-readable storage media includes, but is not limited to, phase change memory (PRAM), static random-access memory (SRAM), dynamic random-access memory (DRAM), RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store the desired information and which can be accessed by the processors 702 of the system 700. In contrast, communication media may embody computer-readable instructions, data structures, program modules, or other data in a modulated data signal, such as a carrier wave, or other transmission mechanism. As defined herein, non-transitory computer-readable storage media do not include communication media.

The computer-readable instructions stored on one or more non-transitory computer-readable storage media, such as the memory 704 and the storage 716, when executed by one or more processors, such as the processors 702, may cause the processors 702 to perform operations described above with reference to FIGS. 1-6. For example, the program modules 712 may include the computer-readable instructions. Generally, computer-readable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes.

The transceiver 706 may include a long-range transceiver 718, a short-range transceiver 720, and/or a wired transceiver 722. The long-range transceiver 718 may include one or more transceivers for communicating over cellular networks, such as 2G, 3G, 4G, 5G, and/or other cellular-wireless access technologies, which may be used to receive various information including tomography data for generating a 3D density, such as the 3D density map 100, hyperspectroscopy mineral composition analysis data, drilling forces applied at core sample locations, etc. from various equipment 724, such as drilling equipment, a hyperspectral analyzer, etc., at a surface mining site 726 or through a data center 728, and to transmit instructions including the one or more drilling locations to the various equipment 724 or through the data center 728 as described above with reference to FIGS. 1-6. The short-range transceiver 720 may include one or more transceivers for communicating using short-range wireless communication schemes, such as Bluetooth, WiFi, ZigBee, NFC, and other communication methods. The short-range transceiver 720 and the wired transceiver 722 may also be used to receive and transmit the same or similar data, information, and instructions as the long-range transceiver 718.

The UI 708 may include input device(s) 730 such as a keyboard, a mouse, a touch-sensitive display, voice input device, etc. Output device(s) 732 such as a display, speakers, a printer, etc. may also be included. For example, the system 700 may display on the display the 3D density map 100, core sample locations, mineral composition analysis results and drilling forces applied from various core sample locations, the 3D mineral composition map 300, and information as described above with reference to FIGS. 1-6.

### INDUSTRIAL APPLICABILITY

The present disclosure provides methods and systems for generating a three dimensional (3D) mineral composition map based on a 3D density map generated from muon tomography data. Traditionally, without knowing locations of regions of interest (ROIs) subsurface possibly containing mineral compositions of interest, a number of holes over a mining site is drilled to locate and determine mineral compositions at various depths. Core samples are extracted from various areas of the surface mining site and the core samples are sent out a laboratory for analysis. This traditional process is time and resource consuming. The 3D density map can identify one or more ROIs which are likely to contain mineral compositions of interest. Based on locations of the ROIs, only locations of core sample holes reaching the ROIs for obtaining core samples may be determined. The core samples may be analyzed in real-time, and time and resource may be substantially reduced. For example, mineral contents may be analyzed in real-time by performing hyperspectral analysis on drilled particles exiting a drilled hole during the drilling process to obtain the core sample.

The system may generate a 3D density map of a surface mining site subsurface based on muon tomography and determine bounding surfaces around a region of interest (ROI) in the 3D density map. The bounding surfaces may be determined based on density ranges selected for the ROI, or historical density ranges associated with expected mineral compositions, and sub-bounding surfaces within the ROI may also be determined. The system may then determine a plurality of drilling locations based on the ROI, which may be selected from a predetermined grid for blasthole drilling of the surface mining site. The system may determine mineral compositions of core samples obtained from core sample locations within the ROI and/or sub-ROIs in real-time utilizing hyperspectroscopy. The system may then generate a 3D mineral composition map based on the 3D density map and the mineral compositions of the core samples.

Unless explicitly excluded, the use of the singular to describe a component, structure, or operation does not exclude the use of plural such components, structures, or operations or their equivalents. The use of the terms “a” and “an” and “the” and “at least one” or the term “one or more,” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B” or one or more of A and B”) is to be construed to mean one item selected from the listed items (A or B) or any combination of two or more of the listed items (A and B; A, A and B; A, B and B), unless otherwise indicated herein or clearly contradicted by context. Similarly, as used herein, the word "or" refers to any possible permutation of a set of items. For example, the phrase "A, B, or C" refers to at least one of A, B, C, or any combination thereof, such as any of: A; B; C; A and B; A and C; B and C; A, B, and C; or multiple of any item such as A and A; B, B, and C; A, A, B, C, and C; etc.

While aspects of the present disclosure have been particularly shown and described with reference to the examples above, it will be understood by those skilled in the art that various additional embodiments may be contemplated by the modification of the disclosed devices, systems, and methods without departing from the spirit and scope of what is disclosed. Such embodiments should be understood to fall within the scope of the present disclosure as determined based upon the claims and any equivalents thereof.

What is claimed is:

1. A method for generating a three dimensional (3D) mineral composition map, the method comprising:
generating a 3D density map based on tomography;
determining bounding surfaces around a region of interest (ROI) in the 3D density map;
determining a plurality of drilling locations based on the ROI;
determining mineral compositions of core samples obtained from core sample locations, the core sample locations associated with the plurality of drilling locations; and
generating a 3D mineral composition map based on the 3D density map and the mineral compositions of the core samples.

2. The method of claim 1, wherein generating the 3D density map based on tomography includes utilizing cosmic ray muon (CRM) tomography.

3. The method of claim 1, wherein determining the plurality of drilling locations based on the ROI includes:
selecting the plurality of drilling locations from a predetermined grid for blasthole drilling.

4. The method of claim 1, wherein determining the bounding surfaces includes determining the bounding surfaces based on at least one of:
density ranges selected for the ROI, or
historical density ranges associated with expected mineral compositions.

5. The method of claim 4, wherein determining the bounding surfaces includes:
determining sub-bounding surfaces within the ROI.

6. The method of claim 1, wherein the core samples are obtained by:
at each location of the plurality of drilling locations:
drilling to a corresponding depth associated with each core sample location within the ROI, and
obtaining a core sample at a preselected depth.

7. The method of claim 6, wherein determining the mineral compositions of the core samples includes:
analyzing the core samples in real-time utilizing hyperspectral analysis; and
determining the mineral compositions of the core samples.

8. The method of claim 7, further comprising:
at each core sample location, determining whether the mineral compositions of the core sample matches expected mineral compositions of the core sample location within an acceptable composition range;
calculating a composition confidence level based on a number of core samples with mineral compositions matching the expected mineral compositions; and
based on the composition confidence level being greater than or equal to a threshold composition confidence level, assigning the expected mineral compositions to the ROI.

9. The method of claim 8, further comprising:
based on the composition confidence level being less than the threshold composition confidence level:
determining one or more drilling locations different from the plurality of drilling locations based on the ROI; and
at each drilling location of the one or more drilling locations, determining mineral compositions of a new core sample obtained at a preselected depth within the ROI in real-time.

10. The method of claim 3, further comprising:
at each blasthole location:
monitoring a drilling force applied for drilling at a corresponding depth, and
determining whether the drilling force applied is within an acceptable drilling force range for expected mineral compositions at the corresponding depth;
calculating a drilling force confidence level based on a number of the drilling forces applied matching corresponding expected drilling forces; and
based on the drilling force confidence level being greater than or equal to a threshold drilling force confidence level, assigning the expected mineral compositions to the ROI.

11. The method of claim 10, further comprising:
based on the drilling force confidence level being less the threshold drilling force confidence level:
determining one or more drilling locations different from the plurality of drilling locations based on the ROI; and
at each drilling location of the one or more drilling locations:
monitoring a drilling force applied for drilling at a preselected depth, and
determining whether the drilling force applied is within the acceptable drilling force range for the expected mineral compositions.

12. A system comprising:
one or more processors; and
a memory coupled to the one or more processors, the memory storing computer-readable instructions that, when executed by the one or more processors, cause the one or more processors to perform operations, the operations comprising:
generating a 3D density map based on cosmic ray muon (CRM) tomography;
determining bounding surfaces around a region of interest (ROI) in the 3D density map based on at least one of:
density ranges selected for the ROI, or
historical density ranges associated with expected mineral compositions;
determining a plurality of drilling locations based on the ROI;
determining mineral compositions of core samples obtained from core sample locations, the core sample locations associated with the plurality of drilling locations in real-time utilizing hyperspectral analysis; and
generating a 3D mineral composition map based on the 3D density map and the mineral compositions of the core samples.

13. The system of claim 12, wherein determining the bounding surfaces includes:
determining the bounding surfaces is based on at least one of:
density ranges selected for the ROI, or
historical density ranges associated with expected mineral compositions, and
determining sub-bounding surfaces within the ROI.

14. The system of claim 12, wherein determining the plurality of drilling locations based on the ROI includes:
selecting the plurality of drilling locations from a predetermined grid for blasthole drilling.

15. The system of claim 12, wherein the operations further comprise:
at each core sample location associated with the plurality of drilling locations, determining whether mineral compositions of a corresponding core sample matches expected mineral compositions of the core sample location within an acceptable composition range;
calculating a composition confidence level based on a number of core samples with mineral compositions matching the expected mineral compositions;
based on the composition confidence level being greater than or equal to a threshold composition confidence level, assigning the expected mineral compositions to the ROI; and
based on the composition confidence level being less than the threshold composition confidence level:
determining one or more drilling locations different from the plurality of drilling locations based on the ROI, and
at each drilling location of the one or more drilling locations, determining mineral compositions of a new core sample obtained at a preselected depth within the ROI in real-time.

16. The system of claim 12, wherein the operations further comprise:
at each core sample location:
monitoring a drilling force applied for drilling at a corresponding depth, and
determining whether the drilling force applied is within an acceptable drilling force range for expected mineral compositions at the corresponding depth;
calculating a drilling force confidence level based on a number of the drilling forces applied matching corresponding expected drilling forces;
based on the drilling force confidence level being greater than or equal to a threshold drilling force confidence level, assigning the expected mineral compositions to the ROI; and
based on the drilling force confidence level being less the threshold drilling force confidence level:
determining one or more drilling locations different from the plurality of drilling locations based on the ROI; and
at each drilling location of the one or more drilling locations:
monitoring a drilling force applied for drilling at a preselected depth, and
determining whether the drilling force applied is within the acceptable drilling force range for the expected mineral compositions.

17. Non-transitory computer-readable storage media storing computer-readable instructions that, when executed by one or more processors, cause the one or more processors to perform operations, the operations comprising:
generating a 3D density map based on cosmic ray muon (CRM) tomography;
determining bounding surfaces around a region of interest (ROI) in the 3D density map based on at least one of:
density ranges selected for the ROI, or
historical density ranges associated with expected mineral compositions;
determining a plurality of drilling locations based on the ROI;
determining mineral compositions of core samples obtained from core sample locations, the core sample locations associated with the plurality of drilling locations in real-time utilizing hyperspectral analysis; and
generating a 3D mineral composition map based on the 3D density map and the mineral compositions of the core samples.

18. The non-transitory computer-readable storage media of claim 17, wherein determining the bounding surfaces includes:
determining the bounding surfaces is based on at least one of:
density ranges selected for the ROI, or
historical density ranges associated with expected mineral compositions, and
determining sub-bounding surfaces within the ROI.

19. The non-transitory computer-readable storage media of claim 17, wherein the operations further comprise:
at each core sample location associated with the plurality of drilling locations, determining whether mineral compositions of a corresponding core sample matches expected mineral compositions of the core sample location within an acceptable composition range;
calculating a composition confidence level based on a number of core samples with mineral compositions matching the expected mineral compositions;
based on the composition confidence level being greater than or equal to a threshold composition confidence level, assigning the expected mineral compositions to the ROI; and
based on the composition confidence level being less than the threshold composition confidence level:
determining one or more drilling locations different from the plurality of drilling locations based on the ROI, and
at each drilling location of the one or more drilling locations, determining mineral compositions of a new core sample obtained at a preselected depth within the ROI in real-time.

20. The non-transitory computer-readable storage media of claim 17, wherein the operations further comprise:
at each core sample location:
monitoring a drilling force applied for drilling at a corresponding depth, and
determining whether the drilling force applied is within an acceptable drilling force range for expected mineral compositions at the corresponding depth;
calculating a drilling force confidence level based on a number of the drilling forces applied matching corresponding expected drilling forces;
based on the drilling force confidence level being greater than or equal to a threshold drilling force confidence level, assigning the expected mineral compositions to the ROI; and
based on the drilling force confidence level being less the threshold drilling force confidence level:
determining one or more drilling locations different from the plurality of drilling locations based on the ROI; and
at each drilling location of the one or more drilling locations:
monitoring a drilling force applied for drilling at a preselected depth, and
determining whether the drilling force applied is within the acceptable drilling force range for the expected mineral compositions.
